(19) United States
(12) Patent Application Publication
(10) Pub. No.: US 2014/0194855 A1
(43) Pub. Date: Jul. 10, 2014

(54) Intravenous Therapy System and Method

(71) Applicants: Abdallah K Kayyal, Beirut (LB); Talal A Kayyal, Houston, TX (US)

(72) Inventors: Abdallah K Kayyal, Beirut (LB); Talal A Kayyal, Houston, TX (US)

(21) Appl. No.: 13/734,921

(22) Filed: Jan. 4, 2013

(51) Int. Cl. A61J 1/10; A61J 1/14; A61M 5/00

(52) U.S. Cl. CPC A61J 1/10; A61M 5/00; A61J 1/14
USPC 604/523

(57) ABSTRACT

An improved intravenous therapy system is disclosed. Said improved intravenous therapy system comprising an opaque shell and an opaque line shell. Said opaque shell wrapped around an IV compartment. Said opaque line shell wrapped around an IV line. Said IV compartment comprises an IV fluid. Said IV line delivers said IV fluid to a patient.

### BACKGROUND

This disclosure relates generally to an improved intravenous therapy system and method. Examples of IV improvements include US Patent Application Numbers US 2008/0139076 A or US 2010/0274214 A1; as well as U.S. Pat. No. 5,776,105. However, none of the above inventions, applications and/or patents, taken either singularly or in combination, is seen to describe the instant disclosure as claimed. Accordingly, an improved intravenous therapy system and method would be advantageous.

### SUMMARY

A plurality of embodiments of an improved intravenous therapy system are disclosed.

First, said improved intravenous therapy system comprising an opaque shell and an opaque line shell. Said opaque shell wrapped around an IV compartment. Said opaque line shell wrapped around an IV line. Said IV compartment comprises an IV fluid. Said IV line delivers said IV fluid to a patient.

Second an improved intravenous therapy method is disclosed. Said method comprising assembling an IV container and an IV line with an opaque material capable of holding an IV fluid.

Third, an improved intravenous therapy system is disclosed. Said system comprising an IV compartment and an IV line. Said IV compartment comprises an IV fluid. Said IV line delivers said IV fluid to a patient. Said IV compartment and said IV line are opaque.

### BRIEF DESCRIPTION OF THE DRAWINGS

FIGS. 1A and 1B illustrate a perspective overview and perspective detailed view of an IV station 100.

FIGS. 2A, 2B and 2C illustrate a side view, front view and top view of IV container 101.

FIGS. 3A, 3B and 3C illustrate an elevated front side view of said IV container 101, a perspective cross-section overview of a first IV container 101a, and a perspective cross-section overview of a second IV container 101b.

FIGS. 4A, 4B and 4C illustrate an elevated front view, a cross-section overview of a first IV line 102a and a cross-section overview of a second IV line 102b.

FIG. 5 illustrates IV container 101 with a graphic design 501.

FIGS. 6A and 6B illustrate a perspective overview of said IV container 101 with a flap 602 in a closed configuration and an open configuration.

### DETAILED DESCRIPTION

Described herein is an improved intravenous therapy system and method. The following description is presented to enable any person skilled in the art to make and use the invention as claimed and is provided in the context of the particular examples discussed below, variations of which will be readily apparent to those skilled in the art. In the interest of clarity, not all features of an actual implementation are described in this specification. It will be appreciated that in the development of any such actual implementation (as in any development project), design decisions must be made to achieve the designers' specific goals (e.g., compliance with system- and business-related constraints), and that these goals will vary from one implementation to another. It will also be appreciated that such development effort might be complex and time-consuming, but would nevertheless be a routine undertaking for those of ordinary skill in the field of the appropriate art having the benefit of this disclosure. Accordingly, the claims appended hereto are not intended to be limited by the disclosed embodiments, but are to be accorded their widest scope consistent with the principles and features disclosed herein.

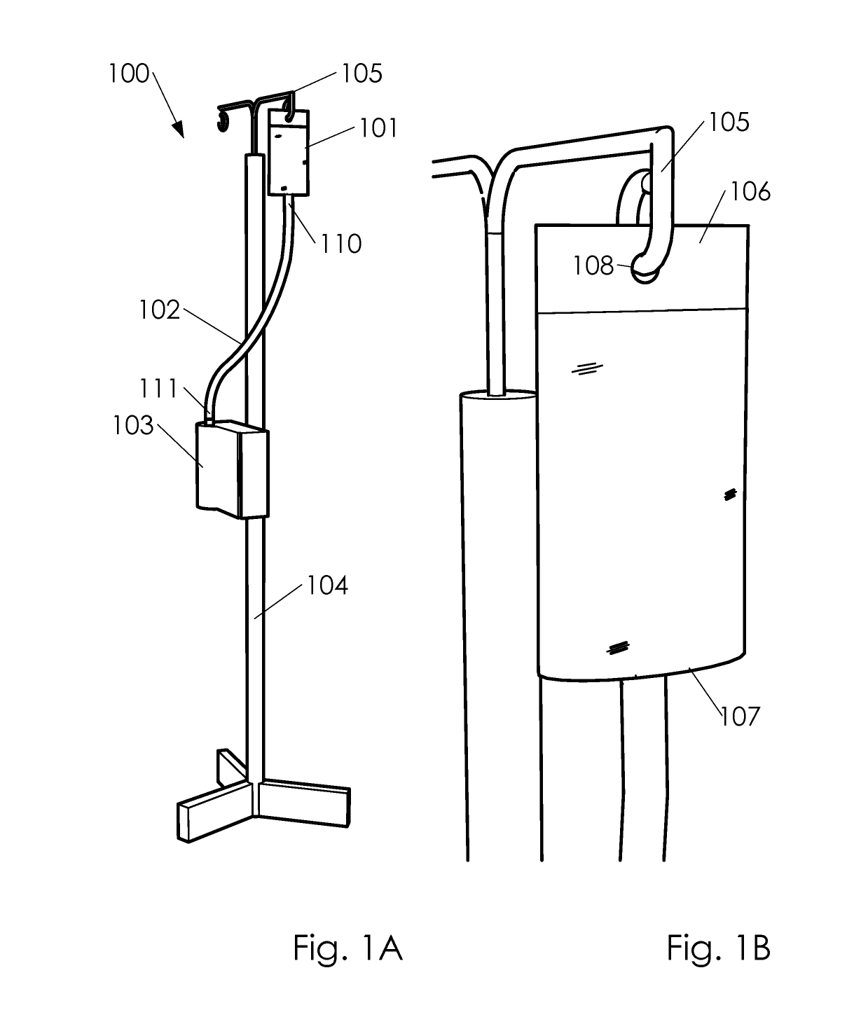

FIGS. 1A and 1B illustrate a perspective overview and perspective detailed view of an IV station 100. IV station 100 can comprise an IV container 101, an IV line 102, an infusion pump 103, a stand 104 and a hook 105. In one embodiment, said IV container 101 can comprise a fluid, as is known in the art. In one embodiment, said infusion pump 103 may not be necessary said fluid of said IV container 101 can flow through said IV line 102 without the aid of said infusion pump 103. In one embodiment, said IV container 101 can be in fluid connection with said infusion pump 103 through said IV line 102. In one embodiment, said hook 105 can attach to a top end of said stand 104. In one embodiment, said IV container 101 can hang from said hook 105. In one embodiment, said IV container 101 can comprise an upper portion 106, an IV compartment 107 and a receptacle 108. In one embodiment, said IV compartment 107 can comprise an IV fluid which can flow through said IV container 101 to said first end 110, said infusion pump 103 and to a patient, as is known in the art. In one embodiment, said IV line 102 can comprise a first end 110 and a second end 111. In one embodiment, said first end 110 can attach to said IV container 101 and said second end 111 can attach to said infusion pump 103. In one embodiment, said IV line 102 can attach directly between said IV container 101 and said patient. In one embodiment, said infusion pump 103 can regulate administration of said IV fluid to said patient. In one embodiment, said IV fluid can comprise one or more volume expanders, one or more blood-based products, one or more blood substitutes, one or more medications, and/or similar.

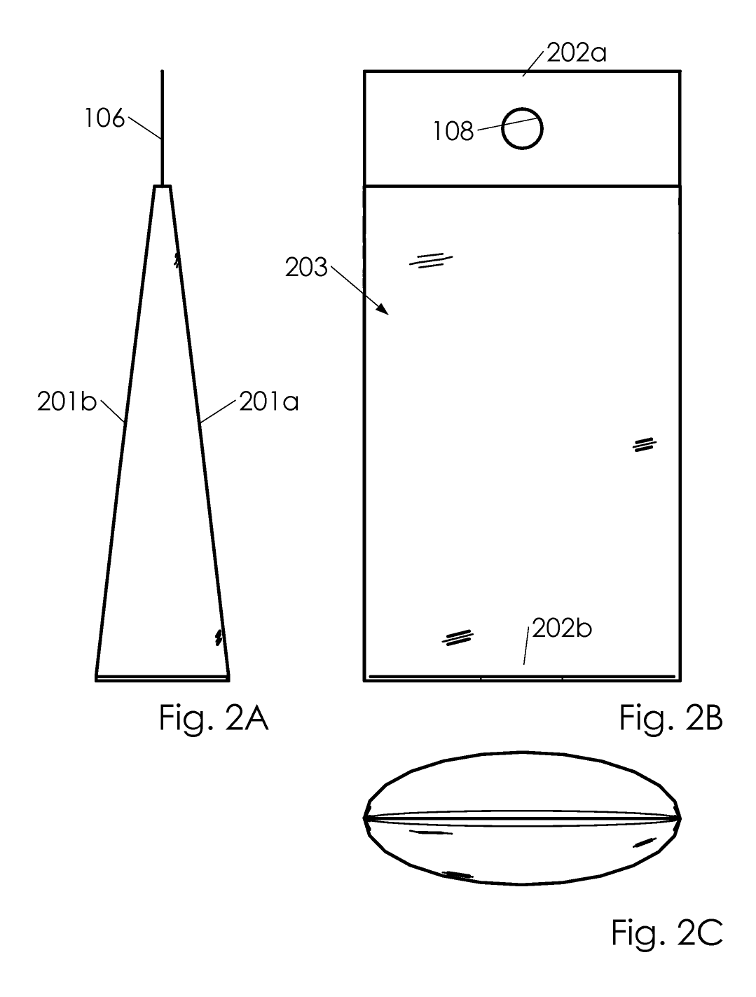

FIGS. 2A, 2B and 2C illustrate a side view, front view and top view of IV container 101. IV container 101 can comprise a first side 201a, a second side 201b, a first end 202a, a second end 202b and an opaque shell 203. In one embodiment, said opaque shell 203 can comprise an external wrapper around an external surface of IV compartment 107. In one embodiment, opaque (meaning not transparent or translucent) can be substantially impervious to light.

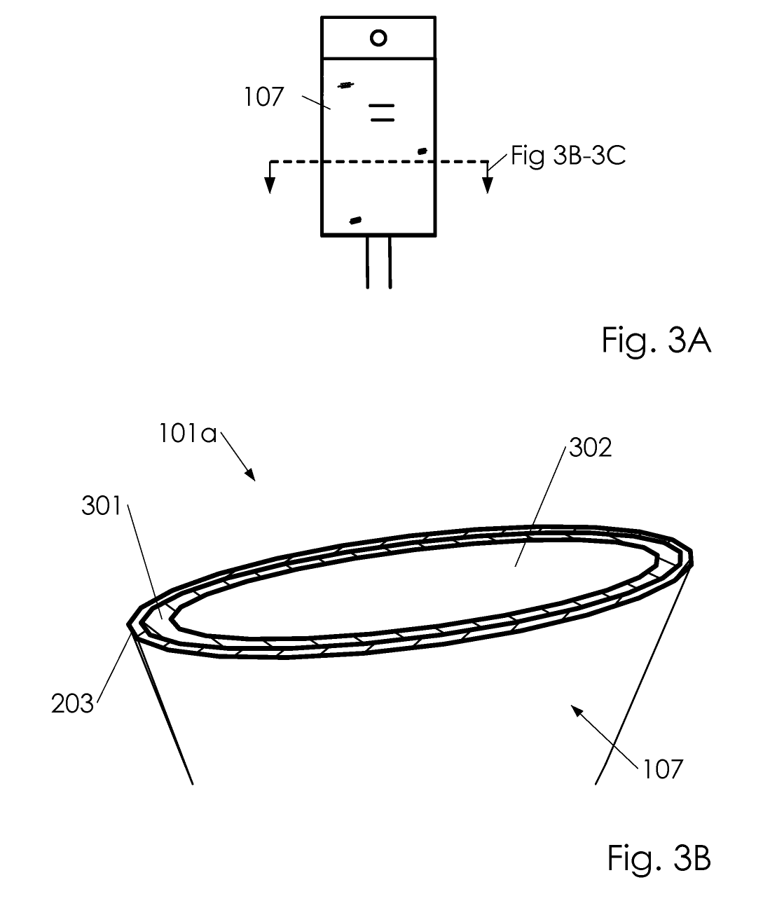

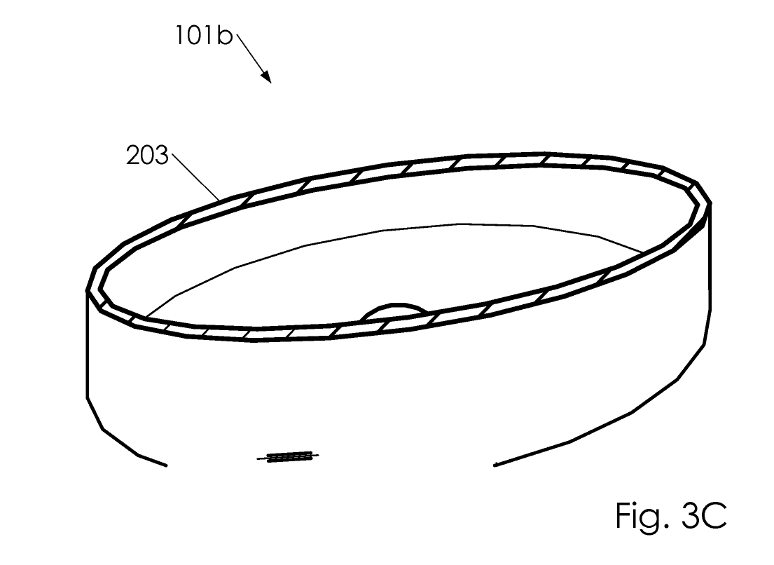

FIGS. 3A, 3B and 3C illustrate an elevated front side view of said IV container 101, a perspective cross-section overview of a first IV container 101a, and a perspective cross-section overview of a second IV container 101b. In one embodiment, said IV compartment 107 can comprise a one or more external layers. In one embodiment, said IV container 101 can comprise multiple embodiments, including said first IV container 101a having a plurality of said one or more external layers and said second IV container 101b having one of said one or more external layers. In one embodiment, said one or more external layers can comprise an internal layer 301 and said opaque shell 203. In one embodiment, said IV compartment 107 can comprise a fluid compartment 302 within said internal layer 301 and said opaque shell 203. In one embodiment, said fluid compartment 302 can comprise a compartment capable of holding said IV fluid and limiting contamination. In one embodiment, said fluid compartment 302 can comprise a plastic bag, or similar. In one embodiment, said opaque shell 203 can comprise aluminum foil. In one embodiment, said opaque shell 203 can comprise photosensitive and UV light protective barrier for said IV fluid within said fluid compartment 302.

In one embodiment, said one or more external layers can comprise a single external layer (as in FIG. 3C) comprising said opaque shell 203; wherein, said opaque shell 203 is capable of containing said IV fluid without said internal layer 301 and capable of substantially blocking light contamination; e.g., an opaque plastic material capable of holding said IV fluid but also opaque. This approach is illustrated in said second IV container 101b. Alternatively, said first IV container 101a demonstrates an embodiment where said one or more external layers comprise a plurality of layers (such as in FIG. 3B).

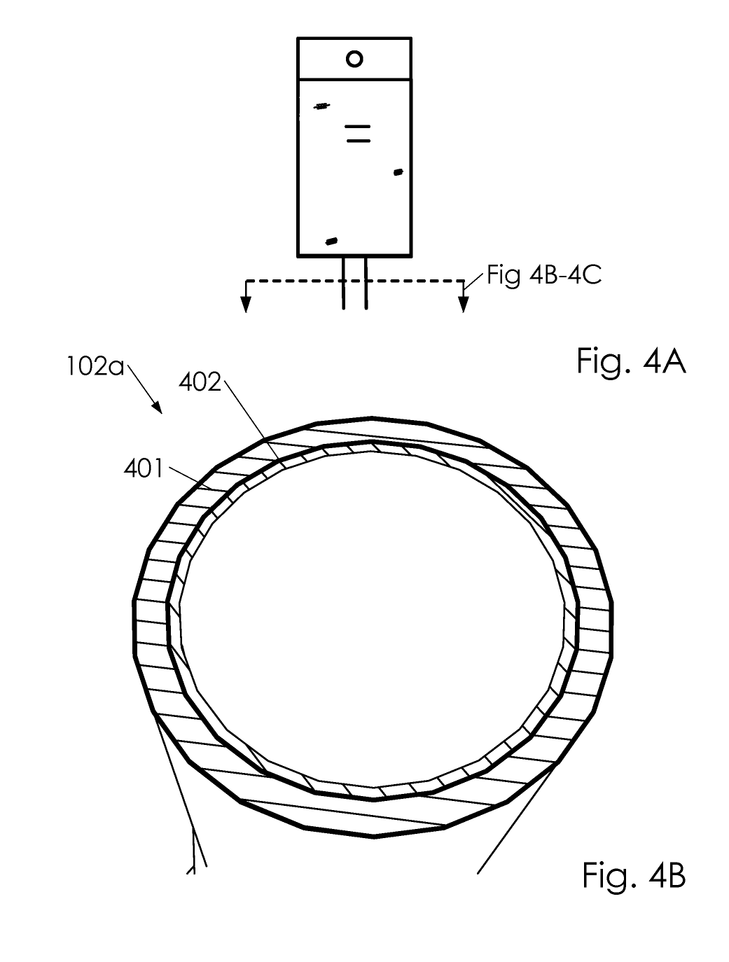

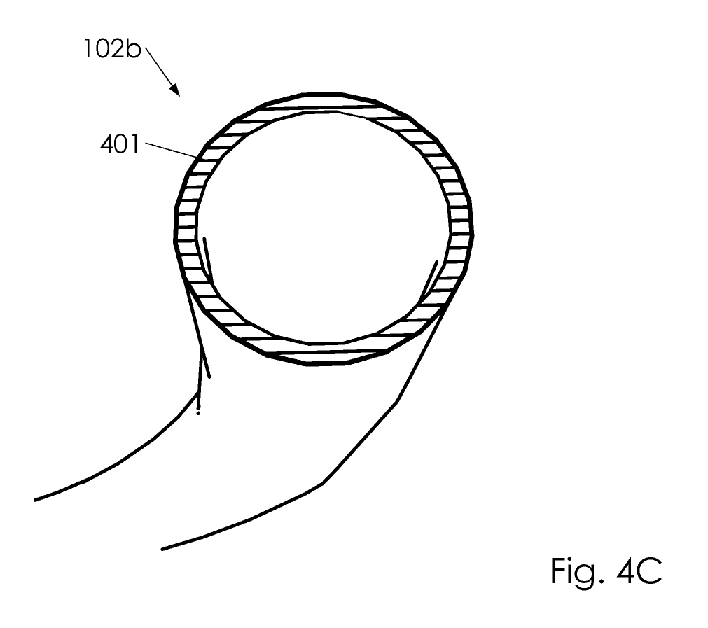

FIGS. 4A, 4B and 4C illustrate an elevated front view, a cross-section overview of a first IV line 102a and a cross-section overview of a second IV line 102b. In one embodiment, said IV line 102 can comprise a one or more external layers, similar to said IV container 101. In one embodiment, said IV line 102 can comprise more than embodiment, such as said first IV line 102a having a plurality of said one or more external layers and said second IV line 102b having one of said one or more external layers. In one embodiment, said IV line 102 can comprise an opaque line shell 401 and a fluid line 402 (as with said first IV line 102a). In another embodiment, said IV line 102 can comprise said opaque line shell 401 and not said fluid line 402 (as with said second IV line 102b). In one embodiment, said fluid line 402 can transmit said IV fluid from said IV container 101 to said infusion pump 103. In one embodiment, said fluid line 402 can comprise a plastic, or similar. In one embodiment, said opaque line shell 401 can comprise light blocking capabilities similar to said opaque shell 203 of said IV container 101.

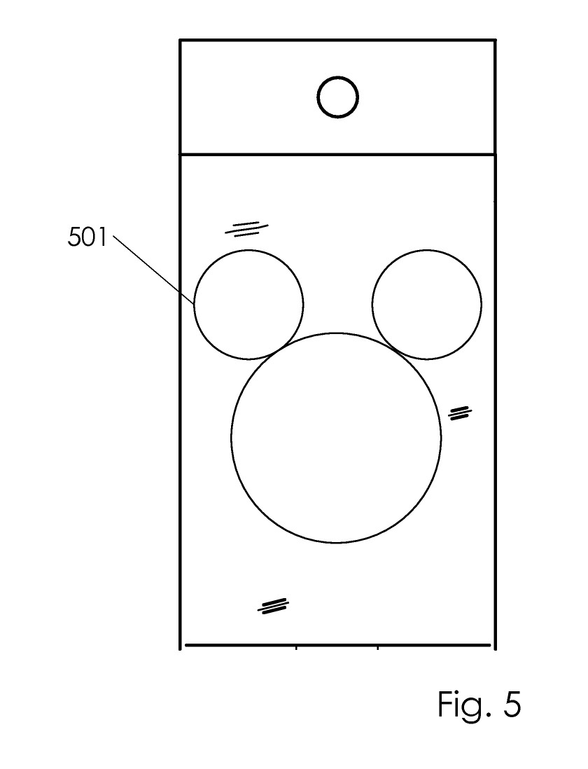

FIG. 5 illustrates IV container 101 with a graphic design 501. In one embodiment, said IV container 101 can comprise a graphic design. In one embodiment, said graphic design 501 can be useful to comfort said patients.

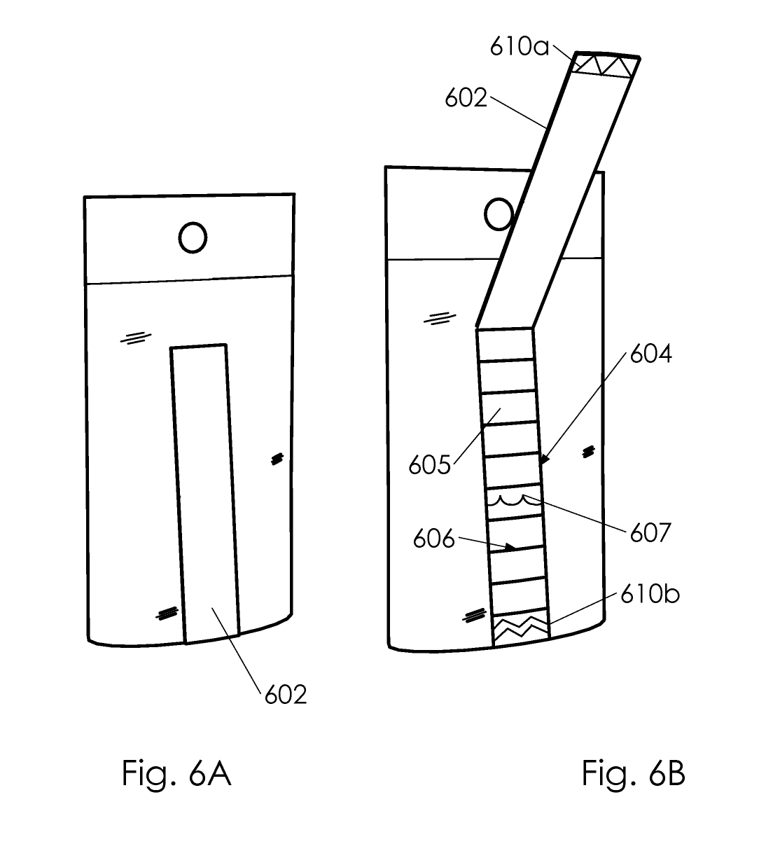

FIGS. 6A and 6B illustrate a perspective overview of said IV container 101 with a flap 602 in a closed configuration and an open configuration. In one embodiment, a portion of opaque shell 203 can comprise an IV volume window 604 selectively covered by said flap 602. In one embodiment, said IV volume window 604 can comprise a transparent portion 605 of said IV container 101. In one embodiment, said IV volume window 604 can comprise a one or more volume markers 606. In one embodiment, said IV volume window 604 can be capable of showing a fluid level 607 by revealing where said IV fluid is in relation to said volume markers 606. In one embodiment, said IV volume window 604 and said flap 602 can comprise a set of hook-and-loop fasteners having a first fastener 610a and a second fastener 610b. In one embodiment, said set of hook-and-loop fasteners can comprise Velcro®. In one embodiment, said first fastener 610a can attach to a portion of said flap 602, and said second fastener 610b can attach to a portion of said IV volume window 604; wherein, said flap 602 can be held in said closed configuration by attaching said first fastener 610a to said second fastener 610b, and said flap 602 can be released from said closed configuration by detaching said first fastener 610a from said second fastener 610b.

In another embodiment, a scale system can measure a weight of IV container 101 and thereby calculate a volume of IV fluid within IV container 101. In one embodiment, calculating said volume of IV fluid within IV container 101 can comprise multiplying a current weight of IV container 101 by a ratio comprising a volume of IV fluid per pound. Wherein, a volume of said IV fluid within said IV container 101 can be determined without exposing said IV fluid to light by using a flap 602 and IV volume window 604.

Various changes in the details of the illustrated operational methods are possible without departing from the scope of the following claims. Some embodiments may combine the activities described herein as being separate steps. Similarly, one or more of the described steps may be omitted, depending upon the specific operational environment the method is being implemented in. It is to be understood that the above description is intended to be illustrative, and not restrictive. For example, the above-described embodiments may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.”

1. An improved intravenous therapy system comprising:
a IV compartment;
said IV container containing a IV fluid; and
said IV container comprising an opaque shell.

2. The improved intravenous therapy system of claim 1 further comprising
said IV line having a first end and a second end;
said first end of said IV line in fluid connection with said IV container;
said IV line attached to a patient; and
said IV container comprising an opaque line shell.

3. The improved intravenous therapy system of claim 2 wherein
said opaque shell and said opaque line shell comprise a photosensitive barrier protecting said IV fluid.

4. The improved intravenous therapy system of claim 2 wherein
said IV line comprises a one or more external layers;
said one or more external layers comprise said opaque line shell and a fluid line; and
said fluid line contains a portion of said IV fluid between said first end and said second end of said IV line.

5. The improved intravenous therapy system of claim 1 wherein
said IV compartment comprises a one or more external layers;
said one or more external layers comprise said opaque shell and an internal layer; and
said internal layer contains said IV fluid.

6. The improved intravenous therapy system of claim 5 wherein said internal layer comprises a plastic bag.

7. The improved intravenous therapy system of claim 1 wherein said opaque shell of said IV container comprises a graphic design.

8. The improved intravenous therapy system of claim 1 wherein a portion of said opaque shell comprises an IV volume window.

9. The improved intravenous therapy system of claim 8 further comprising a flap covering a portion of said IV volume window.

10. The improved intravenous therapy system of claim 9 wherein,
said flap and said IV volume window comprise a set of hook-and-loop fasteners having a first fastener and a second fastener;
said first fastener is attached to a portion of said flap;
said second fastener is attached to a portion of said IV volume window; and
said set of hook-and-loop fasteners are capable of selectively attaching to one another.

11. The improved intravenous therapy system of claim 8 wherein
said IV volume window comprises a one or more volume markers.

12. The improved intravenous therapy system of claim 1 wherein
said opaque shell and said opaque line shell comprise a UV light barrier protecting said IV fluid.

13. An improved intravenous therapy method comprising:
assembling an IV container and an IV line with an opaque material capable of holding an IV fluid.

14. An improved intravenous therapy system comprising:
a IV compartment;
said IV container containing a IV fluid;
said IV container comprising an opaque shell;
said IV line having a first end and a second end;
said first end of said IV line in fluid connection with said IV container;
said IV line attached to a patient;
said IV container comprising an opaque line shell;
said opaque shell and said opaque line shell comprise a photosensitive barrier protecting said IV fluid.
